(19) United States
(12) Patent Application Publication
(10) Pub. No.: US 2022/0017455 A1
(43) Pub. Date: Jan. 20, 2022

(54) SURFACE TREATMENT AGENT, SURFACE TREATMENT METHOD, AND REGION SELECTIVE FILM FORMATION METHOD FOR SURFACE OF SUBSTRATE

(71) Applicant: TOKYO OHKA KOGYO CO., LTD., Kawasaki-shi (JP)

(72) Inventors: Natsumi Okawa, Kawasaki-shi (JP); Makoto Sato, Kawasaki-shi (JP); Issei Suzuki, Kawasaki-shi (JP); Jun Iioka, Kawasaki-shi (JP)

(21) Appl. No.: 17/304,445

(22) Filed: Jun. 21, 2021

(30) Foreign Application Priority Data
Jul. 14, 2020 (JP) 2020-120730

(51) Int. Cl. C07C 259/06; C09D 5/00; C07C 259/10; C23C 16/02; C23C 16/455; C23C 16/04

(52) U.S. Cl. CPC C07C 259/06; C09D 5/002; C23C 16/04; C23C 16/0281; C23C 16/45525; C07C 259/10

(57) ABSTRACT

A surface treatment agent used for treating a substrate which has a surface having two or more regions made of materials that are different from each other, the agent including a compound (H) represented by Formula (H-1). In the formula, R¹ represents a linear or branched alkyl group having 1 to 30 carbon atoms, a linear or branched fluorinated alkyl group having 1 to 30 carbon atoms, an aromatic hydrocarbon group, or a cycloalkyl group having 3 to 12 carbon atoms, and R² represents a hydrogen atom, a linear or branched alkyl group having 1 to 8 carbon atoms, or a cycloalkyl group having 3 to 12 carbon atoms)

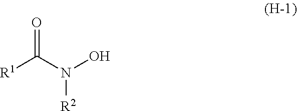

### BACKGROUND OF THE INVENTION

### Field of the Invention

The present invention relates to a surface treatment agent, a surface treatment method, and a region selective film formation method for a surface of a substrate.

Priority is claimed on Japanese Patent Application No. 2020-120730, filed Jul. 14, 2020, the content of which is incorporated herein by reference.

### Description of Related Art

In recent years, with the progress of high integration and miniaturization of semiconductor devices, miniaturization of organic patterns used as masks and inorganic patterns prepared by etching treatments has proceeded, and thus the film thickness needs to be controlled at an atomic layer level.

As a method of forming a film, which is thin at an atomic layer level, on a substrate, an atomic layer deposition method (ALD; hereinafter, also simply referred to as an “ALD method”) is known. The ALD method is known to have both high step coverage and film thickness controllability as compared with a typical chemical vapor deposition (CVD) method.

The ALD method is a thin-film forming technique of alternately supplying two kinds of gases having elements constituting a film intended to be formed as main components onto a substrate and repeatedly forming a thin film a plurality of times on the substrate in an atomic layer unit to form a film having a desired thickness.

In the ALD method, a self-control function (self-limit function) of growth in which only one layer or several layers of raw material gas components are adsorbed on a surface of a substrate while the raw material gases are supplied and the extra raw material gases do not contribute to the growth is used.

For example, in a case where an Al₂O₃ film is formed on a substrate, a raw material gas formed of trimethyl aluminum (TMA) and an oxidizing gas containing 0 are used. Further, in a case where a nitride film is formed on a substrate, a nitride gas is used in place of the oxidizing gas.

In recent years, a region selective film formation method for a surface of a substrate using the ALD method has been attempted (see J. Phys. Chem. C 2014, 118, pp. 10957 to 10962 and ACS NANO Vol. 9, No. 9, pp. 8710 to 8717 (2015)).

Along with such attempts, there has been a demand for a substrate having a region-selectively modified surface so that the substrate can be suitably applied to a region selective film formation method for a substrate according to the ALD method.

In the film forming method, control of the film thickness at an atomic layer level, step coverage, and miniaturization of patterning are expected by using the ALD method.

### CITATION LIST

### Non Patent Documents

[Non Patent Document 1] J. Phys. Chem. C 2014, 118, pp. 10957 to 10962
[Non Patent Document 2] ACS NANO Vol. 9, No. 9, pp. 8710 to 8717 (2015)

### SUMMARY OF THE INVENTION

In the methods described in J. Phys. Chem. C 2014, 118, pp. 10957 to 10962 and ACS NANO Vol. 9, No. 9, pp. 8710 to 8717 (2015), phosphonic acid is used as a material forming a self-assembled monolayer (hereinafter, also referred to as a “SAM agent”) applied to the region selective film formation method for a substrate according to the ALD method. Phosphonic acid has high heat resistance, but has a degraded desorption property from a substrate and as a result, phosphorus elements remain on a surface of the substrate. Therefore, the ALD method has a problem that subsequent atomic deposition may be inhibited. Further, in a case where phosphonic acid is used as the SAM agent, since phosphonic acid shows selectivity for a specific substrate, there is a problem in that the kind of substrate to which the ALD method can be applied can be limited.

The present invention has been made in consideration of the above-described circumstances, and an object thereof is to provide a surface treatment agent having satisfactory properties such as substrate selectivity and the desorption property, a surface treatment method carried out using the surface treatment agent, and a region selective film formation method for a surface of a substrate to which the surface treatment method has been applied, in a method of treating a substrate which has a surface having two or more regions made of materials that are different from each other.

### Solution to Problem

In order to solve the above-described problems, the present invention has adopted the following configurations.

According to a first aspect of the present invention, there is provided a surface treatment agent used for treating a substrate which has a surface having two or more regions made of materials that are different from each other, the agent including: a compound (H) represented by Formula (H-1).

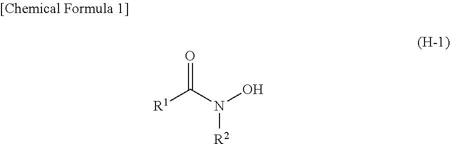

[In the formula, R¹ represents a linear or branched alkyl group having 1 to 30 carbon atoms which may have a substituent, a linear or branched fluorinated alkyl group having 1 to 30 carbon atoms which may have a substituent, an aromatic hydrocarbon group which may have a substituent, or a cycloalkyl group having 3 to 12 carbon atoms which may have a substituent, and R² represents a hydrogen atom, a linear or branched alkyl group having 1 to 8 carbon atoms which may have a substituent, or a cycloalkyl group having 3 to 12 carbon atoms which may have a substituent.]

According to a second aspect of the present invention, there is provided a surface treatment method for a substrate which has a surface having two or more regions made of materials that are different from each other, the method including: exposing the surface to the surface treatment agent according to the first aspect.

According to a third aspect of the present invention, there is provided a region selective film formation method for a surface of a substrate, the method including: treating the surface of the substrate using the surface treatment method according to the second aspect; and forming a film on the surface of the substrate, which has been subjected to the surface treatment, using an atomic layer deposition method, in which an amount of a material of the film to be deposited region-selectively varies.

According to the present invention, it is possible to provide a surface treatment agent having satisfactory properties such as substrate selectivity and the desorption property, a surface treatment method carried out using the surface treatment agent, and a region selective film formation method for a surface of a substrate to which the surface treatment method has been applied, in a method of treating a substrate which has a surface having two or more regions made of materials that are different from each other.

### DETAILED DESCRIPTION OF THE INVENTION

### First Embodiment: Surface Treatment Agent

A surface treatment agent according to a first embodiment of the present invention is a surface treatment agent used for treating a substrate which has a surface having two or more regions made of materials that are different from each other (hereinafter, also simply referred to as a “surface to be treated”).

In the present embodiment, from the viewpoint of ease of application to a region selective film formation method for a surface of a substrate, it is preferable that at least one of the two or more regions in the surface to be treated may have a metal surface.

In the present embodiment, in a case where the surface to be treated has two regions, the surface to be treated has a first region and a second region that is made of a material different from that of the first region and is adjacent to the first region. In such a case, the “proximity regions” are the first region and the second region.

Here, each of the first region and the second region may or may not be divided into a plurality of regions.

In the present embodiment, in a case where the surface to be treated has three or more regions, the surface to be treated has a first region, a second region that is made of a material different from that of the first region and is adjacent to the first region, and a third region that is made of a material different from that of the second region and is adjacent to the second region. In such a case, the “proximity regions” may be the first region and the second region (that is, the adjacent regions) or the first region and the third region (that is, the regions separated by a region).

Further, in a case where the first region and the third region are made of materials that are not different from each other, the “proximity regions” are the first region and the second region, or the second region and the third region (that is, adjacent regions).

Here, each of the first region, the second region, and the third region may or may not be divided into a plurality of regions.

In the present embodiment, the same idea can be applied to a case where the surface to be treated has the fourth or more regions.

The upper limit of the number of regions made of different materials is not particularly limited as long as the effects of the present invention are not impaired, and the upper limit thereof is, for example, 7 or less or 6 or less and typically 5 or less.

The surface treatment agent according to the present embodiment contains a compound (H) represented by Formula (H-1).

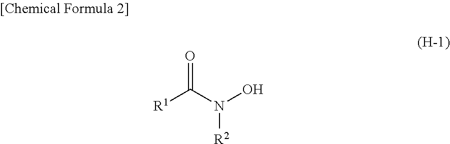

[In the formula, R¹ represents a linear or branched alkyl group having 1 to 30 carbon atoms which may have a substituent, a linear or branched fluorinated alkyl group having 1 to 30 carbon atoms which may have a substituent, an aromatic hydrocarbon group which may have a substituent, or a cycloalkyl group having 3 to 12 carbon atoms which may have a substituent, and R² represents a hydrogen atom, a linear or branched alkyl group having 1 to 8 carbon atoms which may have a substituent, or a cycloalkyl group having 3 to 12 carbon atoms which may have a substituent.]

In Formula (H-1), the linear or branched alkyl group having 1 to 30 carbon atoms as R¹ has preferably 5 to 25 carbon atoms, more preferably 6 to 22 carbon atoms, and still more preferably 7 to 20 carbon atoms.

Specific examples of the linear or branched alkyl group having 1 to 30 carbon atoms as R¹ include a methyl group, an ethyl group, a propyl group, a butyl group, a pentyl group, a hexyl group, a heptyl group, an octyl group, a nonyl group, a decyl group, an undecyl group, a dodecyl group, a tridecyl group, an isotridecyl group, a tetradecyl group, a pentadecyl group, a hexadecyl group, an isohexadecyl group, a heptadecyl group, an octadecyl group, a nonadecyl group, an icosyl group, a henicosyl group, a docosyl group, and isomers of the above-described alkyl groups.

In Formula (H-1), as the linear or branched fluorinated alkyl group having 1 to 30 carbon atoms as R¹, a group in which some or all hydrogen atoms of the linear or branched alkyl group having 1 to 30 carbon atoms have been substituted with fluorine atoms is an exemplary example.

The linear or branched alkyl group having 1 to 30 carbon atoms or the linear or branched fluorinated alkyl group having 1 to 30 carbon atoms as R¹ may have a substituent. Examples of the substituent include a hydroxy group, a carboxy group, a halogen atom (such as a fluorine atom, a chlorine atom, or a bromine atom), an alkoxy group (such as a methoxy group, an ethoxy group, a propoxy group, or a butoxy group), and an alkyloxycarbonyl group.

Examples of the aromatic hydrocarbon group as R¹ include a phenyl group, a naphthyl group, an anthryl group, a p-methylphenyl group, a p-tert-butylphenyl group, a p-adamantylphenyl group, a tolyl group, a xylyl group, a cumenyl group, a mesityl group, a biphenyl group, a phenanthryl group, a 2,6-diethylphenyl group, and a 2-methyl-6-ethylphenyl group. Among these, as the aromatic hydrocarbon group which may have a substituent as R, a phenyl group or a naphthyl group is preferable, and a phenyl group is more preferable.

The aromatic hydrocarbon group as R¹ may have a substituent. Examples of the substituent include an alkyl group, an alkoxy group, a halogen atom, a halogenated alkyl group, a hydroxyl group, a carbonyl group, and a nitro group.

In Formula (H-1), examples of the cycloalkyl group having 3 to 12 carbon atoms as R¹ include a cyclopropyl group, a cyclobutyl group, a cyclopentyl group, a cyclohexyl group, a cycloheptyl group, a cyclooctyl group, a cyclodecyl group, and a cyclododecyl group.

The cycloalkyl group having 3 to 12 carbon atoms as R¹ may have a substituent. Examples of the substituent include an alkyl group, a hydroxy group, a carboxy group, a halogen atom (such as a fluorine atom, a chlorine atom, or a bromine atom), an alkoxy group (such as a methoxy group, an ethoxy group, a propoxy group, or a butoxy group), and an alkyloxycarbonyl group.

Among these, R¹ represents preferably a linear or branched alkyl group having 1 to 30 carbon atoms which may have a substituent or an aromatic hydrocarbon group which may have a substituent and more preferably a linear or branched alkyl group having 5 to 25 carbon atoms from the viewpoint of application to a method of treating a substrate which has a surface having two or more regions made of materials that are different from each other.

In Formula (H-1), examples of the linear or branched alkyl group having 1 to 8 carbon atoms as R² includes a methyl group, an ethyl group, a propyl group, a butyl group, a pentyl group, a hexyl group, a heptyl group, an octyl group, and isomers of the above-described alkyl groups.

The linear or branched alkyl group having 1 to 8 carbon atoms as R² may have a substituent. Examples of the substituent include a hydroxy group, a carboxy group, a halogen atom (such as a fluorine atom, a chlorine atom, or a bromine atom), an alkoxy group (such as a methoxy group, an ethoxy group, a propoxy group, or a butoxy group), and an alkyloxycarbonyl group.

In Formula (H-1), examples of the cycloalkyl group having 3 to 12 carbon atoms as R² include the same groups as those for the cycloalkyl group having 3 to 12 carbon atoms as R¹.

Among these, it is preferable that R² represents a hydrogen atom.

In the present embodiment, the compound (H) may be used alone or in combination of two or more kinds thereof.

In the surface treatment agent according to the present embodiment, the content of the compound (H) is preferably in a range of 1 ppm to 20% by mass, more preferably in a range of 10 ppm to 15% by mass, and still more preferably in a range of 100 ppm to 10% by mass with respect to the total mass of the surface treatment agent.

In a case where the content of the compound (H) is in the above-described preferable range, various properties (such as the heat resistance and the desorption property) required for the method of treating a substrate which has a surface having two or more regions made of materials that are different from each other are likely to be satisfactory.

Water The surface treatment agent according to the present embodiment may contain water in order to further improve the water repellency. Water may contain a trace amount of components that are inevitably mixed. As the water used for the surface treatment agent according to the present embodiment, it is preferable to use water that has been subjected to a purification treatment such as distilled water, ion exchange water, or ultrapure water and more preferable to use ultrapure water typically used for manufacture of semiconductors.

In the surface treatment agent according to the present embodiment, the content of water in a case where the surface treatment agent contains water is preferably in a range of 0.01% to 25% by mass, more preferably in a range of 0.03% to 20% by mass, and still more preferably in a range of 0.05% to 15% by mass.

In a case where the content of water is in the above-described preferable range and in a case where at least one region has a metal surface in the method for treating a substrate which has a surface having two or more regions formed of materials that are different from each other, the compound (H) is likely to be adsorbed on the region having the metal surface, and the selectivity of the surface treatment agent for the region having the metal surface is likely to be improved. Further, the water repellency of the surface treatment agent is likely to be further improved.

Solvent In the present embodiment, it is preferable that the surface treatment agent dissolves each component in a solvent. Since the surface treatment agent contains a solvent, the surface treatment is easily performed on the substrate according to a dipping method, a spin coating method, or the like.

Specific examples of the solvent include sulfoxides such as dimethyl sulfoxide; sulfones such as dimethyl sulfone, diethyl sulfone, bis(2-hydroxyethyl) sulfone, and tetramethylene sulfone; amides such as N,N-dimethylformamide, N-methylformamide, N,N-dimethylacetamide, N-methylacetamide, and N,N-diethylacetamide; lactams such as N-methyl-2-pyrrolidone, N-ethyl-2-pyrrolidone, N-propyl-2-pyrrolidone, N-hydroxymethyl-2-pyrrolidone, and N-hydroxyethyl-2-pyrrolidone; imidazolidinones such as 1,3-dimethyl-2-imidazolidinone, 1,3-diethyl-2-imidazolidinone, and 1,3-diisopropyl-2-imidazolidinones; dialkyl glycol ethers such as dimethyl glycol, dimethyl diglycol, dimethyl triglycol, methyl ethyl diglycol, diethyl glycol, and triethylene glycol butyl methyl ether; monoalcohol-based solvents such as methanol, ethanol, n-propanol, isopropanol, n-butanol, isobutanol, sec-butanol, tert-butanol, n-pentanol, isopentanol, 2-methylbutanol, sec-pentanol, tert-pentanol, 3-methoxybutanol, 3-methyl-3-methoxybutanol, n-hexanol, 2-methylpentanol, sec-hexanol, 2-ethylbutanol, sec-heptanol, 3-heptanol, n-octanol, 2-ethylhexanol, sec-octanol, n-nonyl alcohol, 2,6-dimethyl-4-heptanol, n-decanol sec-undecyl alcohol, trimethylnonyl alcohol, sec-tetradecyl alcohol, sec-heptadecyl alcohol, phenol, cyclohexanol, methylcyclohexanol, 3,3,5-trimethylcyclohexanol, benzyl alcohol, phenyl methyl carbinol, diacetone alcohol, and cresol; (poly)alkylene glycol monoalkyl ethers such as ethylene glycol monomethyl ether, ethylene glycol monoethyl ether, ethylene glycol mono-n-propyl ether, ethylene glycol mono-n-butyl ether, diethylene glycol monomethyl ether, diethylene glycol monoethyl ether, diethylene glycol mono-n-propyl ether, diethylene glycol mono-n-butyl ether, triethylene glycol monomethyl ether, triethylene glycol monoethyl ether, propylene glycol monomethyl ether, propylene glycol monoethyl ether, propylene glycol mono-n-propyl ether, propylene glycol mono-n-butyl ether, dipropylene glycol monomethyl ether, dipropylene glycol monoethyl ether, dipropylene glycol mono-n-propyl ether, dipropylene glycol mono-n-butyl ether, tripropylene glycol monomethyl ether, and tripropylene glycol monoethyl ether; (poly)alkylene glycol monoalkyl ether acetates such as ethylene glycol monomethyl ether acetate, ethylene glycol monoethyl ether acetate, ethylene glycol monobutyl ether acetate, diethylene glycol monomethyl ether acetate, diethylene glycol monoethyl ether acetate, propylene glycol monomethyl ether acetate, and propylene glycol monoethyl ether acetate; other ethers such as dimethyl ether, diethyl ether, methyl ethyl ether, dipropyl ether, diisopropyl ether, dibutyl ether, diisoamyl ether, diethylene glycol dimethyl ether, diethylene glycol methyl ethyl ether, diethylene glycol diethyl ether, tetraethylene glycol dimethyl ether, and tetrahydrofuran; ketones such as methyl ethyl ketone, cyclohexanone, 2-heptanone, and 3-heptanone; lactic acid alkyl esters such as methyl 2-hydroxy propionate and ethyl 2-hydroxy propionate; other esters such as ethyl 2-hydroxy-2-methyl propionate, methyl 3-methoxy propionate, ethyl 3-methoxy propionate, methyl 3-ethoxy propionate, ethyl 3-ethoxy propionate, ethyl ethoxy acetate, ethyl hydroxy acetate, methyl 2-hydroxy-3-methyl butanoate, 3-methoxybutyl acetate, 3-methyl-3-methoxy-1-butyl acetate, 3-methyl-3-methoxybutyl propionate, ethyl acetate, n-propyl acetate, i-propyl acetate, n-butyl acetate, i-butyl acetate, n-pentyl acetate, n-hexyl acetate, n-heptyl acetate, n-octyl acetate, n-pentyl formate, i-pentyl acetate, n-butyl propionate, ethyl butyrate, n-propyl butyrate, i-propyl butyrate, n-butyl butyrate, methyl n-octanoate, methyl decanoate, methyl pyruvate, ethyl pyruvate, n-propyl pyruvate, methyl acetoacetate, ethyl acetoacetate, ethyl 2-oxobutanoate, dimethyl adipate, and propylene glycol diacetate; lactones such as propyrolactone, γ-butyrolactone, and 6-pentyrolactone; linear, branched, or cyclic aliphatic hydrocarbons such as n-hexane, n-heptane, n-octane, n-nonane, methyloctane, n-decane, n-undecane, n-dodecane, 2,2,4,6,6-pentamethylheptane, 2,2,4,4,6,8,8-heptamethylnonane, cyclohexane, and methylcyclohexane; aromatic hydrocarbons such as benzene, toluene, xylene, 1,3,5-trimethylbenzene, and naphthalene; and terpenes such as p-menthane, diphenylmenthane, limonene, terpinene, bornane, norbornane, and pinane.

Among these, as the solvent, 3-methyl-3-methoxy-1-butyl acetate, ethyl acetate, propylene glycol monomethyl ether acetate, propylene glycol monomethyl ether, diethylene glycol monomethyl ether, isopropanol, or methyl ethyl ketone is preferable, and propylene glycol monomethyl ether is more preferable.

Further, since the surface treatment agent according to the present embodiment has a high selectivity particularly for a region having a metal surface, the surface treatment agent can be suitably applied particularly to the region selective film formation method for a surface of a substrate using the ALD method.

### Second Embodiment: Surface Treatment Method

A surface treatment method according to a second embodiment is a surface treatment method for a substrate which has a surface having two or more regions made of materials that are different from each other, the method including exposing the surface to the surface treatment agent according to the first embodiment.

In the surface treatment method according to the present embodiment, the surface has two or more regions, and the contact angles in adjacent regions of the two or more regions made of materials that are different from each other are made to be different from each other by reacting the compound (H) with the two or more regions. In the present embodiment, from the viewpoint of ease of application to the region selective film formation method for a surface of a substrate, it is preferable that at least one of the two or more regions has a metal surface.

In the present embodiment, examples of the “substrate” to be subjected to a surface treatment include substrates used for preparing semiconductor devices, and specific examples thereof include a silicon (Si) substrate, a silicon nitride (SiN) substrate, a silicon oxide film (Ox) substrate, a tungsten (W) substrate, a cobalt (Co) substrate, a titanium nitride (TiN) substrate, a tantalum nitride (TaN) substrate, a germanium (Ge) substrate, a silicon germanium (SiGe) substrate, an aluminum (Al) substrate, a nickel (Ni) substrate, a ruthenium (Ru) substrate, and a copper (Cu) substrate.

The “surface of a substrate” may indicate a surface of an inorganic pattern or an organic pattern provided on a substrate, a surface of an unpatterned inorganic layer or an unpatterned organic layer provided on a substrate, or the like, in addition to a surface of a substrate itself.

Examples of the inorganic pattern provided on a substrate include a pattern formed by etching a surface of an inorganic layer present on a substrate according to a photoresist method to prepare a mask and performing an etching treatment thereon. Examples of the inorganic layer include an oxide film of an element constituting a substrate; and a film or a layer of an inorganic substance such as SiN, Ox, W, Co, TiN, TaN, Ge, SiGe, Al, Al₂O₃, Ni, Ru, or Cu formed on a surface of a substrate, in addition to a substrate itself.

Such a film or layer is not particularly limited, and examples thereof include a film or a layer of an inorganic substance formed in the process of preparation of a semiconductor device.

Examples of the organic pattern provided on a substrate include a resin pattern formed on a substrate using a photoresist or the like according to a photolithography method. Such an organic pattern can be formed by, for example, forming an organic layer which is a photoresist film on a substrate, exposing the organic layer through a photomask, and developing the organic layer. The organic layer may be an organic layer provided on a surface of a laminated film provided on a surface of a substrate in addition to a surface of a substrate itself. Such an organic layer is not particularly limited, and examples thereof include a film of an organic substance provided to form a mask by performing etching in the process of preparation of a semiconductor device.

(Embodiment in which Surface of Substrate has Two Regions)

In the surface treatment method according to the second embodiment, the surface of the substrate has two or more regions, and proximity regions of the two or more regions are made of materials that are different from each other.

Among the two or more regions, as a region where the contact angle of water tends to be larger (preferably, the surface free energy is smaller) than that of other regions, a region containing at least one selected from the group consisting of W, Co, Al, Al₂O₃, Ni, Ru, Cu, TiN, and TaN is an exemplary example.

Among the two or more regions, as a region where the contact angle of water tends to be smaller (preferably, the surface free energy is larger) than that of other regions, a region containing at least one selected from the group consisting of Si, Al₂O₃, SiN, Ox, TiN, TaN, Ge, and SiGe is an exemplary example.

In the present embodiment, in a case where the surface of the substrate has two regions, the surface of the substrate has a first region and a second region that is formed of a material different from that of the first region and is adjacent to the first region. In such a case, the “proximity regions” are the first region and the second region. Here, each of the first region and the second region may or may not be divided into a plurality of regions.

Examples of the first region and the second region include an embodiment in which the surface of the substrate itself is set as the first region and a surface of an inorganic layer formed on the surface of the substrate is set as the second region and an embodiment in which a surface of a first inorganic layer formed on the surface of the substrate is set as the first region and a surface of a second inorganic layer formed on the surface of the substrate is set as the second region. Further, an embodiment in which an organic layer is formed in place of the formation of these inorganic layers can also be an exemplary example.

As the embodiment in which the surface of the substrate itself is set as the first region and a surface of an inorganic layer formed on the surface of the substrate is set as the second region, from the viewpoint of selectively improving the hydrophobicity to improve a difference in contact angle of water between two or more regions adjacent to each other and formed of materials that are different from each other on the surface of the substrate, an embodiment in which a surface of at least one substrate selected from the group consisting of a Si substrate, a SiN substrate, an Ox substrate, a TiN substrate, a TaN substrate, a Ge substrate, and a SiGe substrate is set as the first region and a surface of an inorganic layer which is formed on the surface of the substrate and contains at least one selected from the group consisting of W, Co, Al, Ni, Ru, Cu, TiN, and TaN is set as the second region is preferable.

Further, as the embodiment in which a surface of a first inorganic layer formed on the surface of the substrate is set as the first region and a surface of a second inorganic layer formed on the surface of the substrate is set as the second region, from the viewpoint of selectively improving the hydrophobicity to improve a difference in contact angle of water between two or more regions adjacent to each other and formed of materials that are different from each other on the surface of the substrate, an embodiment in which a surface of a first inorganic layer which is formed on a surface of an optional substrate (for example, a Si substrate) and contains at least one selected from the group consisting of SiN, Ox, TiN, TaN, Ge, and SiGe is set as the first region and a surface of a second inorganic layer which is formed on the surface of the substrate and contains at least one selected from the group consisting of W, Co, Al, Ni, Ru, Cu, TiN, and TaN is set as the second region is preferable.

(Embodiment in which Surface of Substrate has Three or More Regions)

In the present embodiment, in a case where the surface of the substrate has three or more regions, the surface of the substrate has a first region, a second region that is made of a material different from that of the first region and is adjacent to the first region, and a third region that is made of a material different from that of the second region and is adjacent to the second region. In such a case, the “proximity regions” may be the first region and the second region (that is, the adjacent regions) or the first region and the third region (that is, the regions separated by a region).

Further, in a case where the first region and the third region are made of materials that are not different from each other, the “proximity regions” are the first region and the second region, or the second region and the third region (that is, adjacent regions).

Here, each of the first region, the second region, and the third region may or may not be divided into a plurality of regions.

Examples of the first region, the second region, and the third region include an embodiment in which the surface of the substrate itself is set as the first region, a surface of a first inorganic layer formed on the surface of the substrate is set as the second region, and a surface of a second inorganic layer formed on the surface of the substrate is set as the third region. Further, an embodiment in which an organic layer is formed in place of the formation of these inorganic layers can also be an exemplary example. Further, an embodiment including both an inorganic layer and an organic layer formed by changing only one of a second inorganic layer and a third inorganic layer to an organic layer can also be an exemplary example.

From the viewpoint of selectively improving the hydrophobicity to improve a difference in contact angle of water between two or more regions adjacent to each other and formed of materials that are different from each other on the surface of the substrate, an embodiment in which a surface of an optional substrate (for example, a Si substrate) is set as the first region, a surface of a first inorganic layer which is formed on the surface of the substrate and contains at least one selected from the group consisting of SiN, Ox, TiN, TaN, Ge, and SiGe is set as the second region, and a surface of a second inorganic layer which is formed on the surface of the substrate and contains at least one selected from the group consisting of W, Co, Al, Ni, Ru, Cu, TiN, and TaN is set as the third region is preferable.

In the present embodiment, the same idea can be applied to a case where the surface of the substrate has the fourth or more regions.

The upper limit of the number of regions made of different materials is not particularly limited as long as the effects of the present invention are not impaired, and the upper limit thereof is, for example, 7 or less or 6 or less and typically 5 or less.

(Exposure) As a method of exposing the surface of the substrate to the surface treatment agent, a method of applying a surface treatment agent (typically a surface treatment agent in a liquid state) which may contain a solvent to the surface of the substrate using a coating method such as a dipping method, a spin coating method, a roll coating method, or a doctor blade method and exposing the surface is an exemplary example.

The exposure temperature is, for example, 10° C. or higher and 90° C. or lower, preferably 20° C. or higher and 80° C. or lower, more preferably 20° C. or higher and 70° C. or lower, and still more preferably 20° C. or higher and 65° C. or lower. From the viewpoint of selectively improving the hydrophobicity between two or more regions adjacent to each other and formed of materials that are different from each other on the surface of the substrate, the exposure time is preferably 20 seconds or longer, more preferably 30 seconds or longer, and still more preferably 45 seconds or longer.

The upper limit of the exposure time is not particularly limited, but is, for example, preferably 2 hours or shorter, more preferably 1.5 hours or shorter, and still more preferably 1.2 hours or shorter.

After the exposure, the surface may be washed (washed with water, an activator rinse, or the like) and/or dried (dried by nitrogen blow or the like) as necessary. For example, as the washing treatment performed on the surface of the substrate having an inorganic pattern or an organic pattern using a washing liquid, a washing liquid of the related art which has been used for a washing treatment of an inorganic pattern or an organic pattern can be employed. Further, examples of the inorganic pattern include SPM (sulfuric acid/hydrogen peroxide water) and APM (ammonia/hydrogen peroxide water), and examples of the organic pattern include water and an activator rinse.

Further, the treated substrate after being dried may be additionally subjected to a heat treatment at 100° C. or higher and 300° C. or lower as necessary.

By exposing the surface, the compound (H) can be region-selectively adsorbed according to the material of each region on the surface of the substrate.

The contact angle of the surface of the substrate with water after the exposure of the surface to the surface treatment agent can be set to be, for example, 40° or greater and 1400 or less.

The upper limit of the contact angle is not particularly limited, but is, for example, 140° or less and typically 130° or less.

In the surface treatment method according to the present embodiment, since the materials of two or more proximity regions on the surface of the substrate are different from each other, it is possible to selectively improve the hydrophobicity between the two or more proximity regions and make the contact angles of water different from each other by exposing the surface.

The difference in contact angle of water between the two or more proximity regions is not particularly limited as long as the effects of the present invention are not impaired and is, for example, 100 or greater. From the viewpoint of selectively improving the hydrophobicity between the two or more proximity regions, the difference in contact angle of water is preferably 20° or greater, more preferably 30° or greater, and still more preferably 40° or greater.

The upper limit of the difference in contact angle is not particularly limited as long as the effects of the present invention are not impaired, and is, for example, 80° or less or 70° or less and typically 60° or less.

### Third Embodiment: Region Selective Film Formation Method for Substrate

Next, a region selective film formation method for a substrate using the surface treatment method according to the second embodiment will be described.

In the present embodiment, the region selective film formation method for the substrate includes treating the surface of the substrate using the surface treatment method according to the second embodiment and forming a film on the surface of the substrate, which has been subjected to the surface treatment, using an atomic layer deposition method (ALD method), in which the amount of the material of the film to be deposited region-selectively varies.

As a result of the surface treatment using the method according to the second embodiment, the contact angles (preferably the surface free energy) of water between the two or more regions are different from each other. In the present embodiment, the amounts of the material forming the film to be deposited between the two or more regions can be made to be region-selectively different from each other on the surface of the substrate.

Specifically, in a region where the contact angle of water between the two or more regions is larger (preferably, the surface free energy is smaller) than that of other regions, the film forming material according to the ALD method is unlikely to be adsorbed (preferably chemical adsorption) on the region on the surface of the substrate, and thus a difference in amount of the film forming material to be deposited is generated between the two or more regions. As a result, it is preferable that the amounts of the film forming material to be deposited on the substrate are region-selectively different from each other.

Examples of the chemical adsorption include chemical adsorption with a hydroxyl group.

Between the two or more regions, as a region where the contact angle of water tends to be greater (preferably, the surface free energy tends to be smaller) than that of other regions, a region containing at least one selected from the group consisting of W, Co, Al, Al₂O₃, Ni, Ru, Cu, TiN, and TaN is an exemplary example.

Among the two or more regions, as a region where the contact angle of water tends to be smaller (preferably, the surface free energy is larger) than that of other regions, a region containing at least one selected from the group consisting of Si, Al₂O₃, SiN, Ox, TiN, TaN, Ge, and SiGe is an exemplary example.

(Film formation according to ALD method) The film forming method according to the ALD method is not particularly limited, and a thin-film forming method carried out by adsorption (preferably chemical adsorption) using at least two gas phase reactants (hereinafter, simply referred to as “precursor gas”) is preferable.

Specifically, a method including the following steps (a) and (b) and repeating the following steps (a) and (b) at least once (one cycle) until a desired film thickness is obtained is an exemplary example.

The step (a) is a step of exposing the substrate subjected to the surface treatment by the method according to the second embodiment to a pulse of a first precursor gas; and the step (B) is a step of exposing the substrate to a pulse of a second precursor gas after the step (a).

The method may include a plasma treatment step and a step of removing or exhausting (purging) the first precursor gas and a reactant thereof with a carrier gas, the second precursor gas, or the like after the step (a) and before the step (b).

The method may or may not include a plasma treatment step and a step of removing or purging the second precursor gas and a reactant thereof with a carrier gas or the like after the step (b).

Examples of the carrier gas include an inert gas such as nitrogen gas, argon gas, or helium gas.

It is preferable that each pulse for each cycle and each layer to be formed are self-controlled and more preferable that each layer to be formed is a monoatomic layer. The film thickness of the monoatomic layer can be set to be, for example, 5 nm or less, preferably 3 nm or less, more preferably 1 nm or less, and still more preferably 0.5 nm or less.

Examples of the first precursor gas include an organic metal, a metal halide, and a metal oxide halide, and specific examples thereof include tantalum pentaethoxide, tetrakis(dimethylamino) titanium, pentakis(dimethylamino) tantalum, tetrakis(dimethylamino) zirconium, tetrakis(dimethylamino) hafnium, tetrakis(dimethylamino) silane, copper hexafluoroacetylacetonate vinyltrimethylsilane, Zn(C₂H₅)₂, Zn(CH₃)₂, TMA (trimethylaluminum), TaCl₅, WF₆, WOCl₄, CuCl, ZrCl₄, AlCl₃, TiCl₄, SiCl₄, and HfCl₄.

Examples of the second precursor gas include a precursor gas capable of decomposing the first precursor and a precursor gas capable of removing the ligand of the first precursor, and specific examples thereof include H₂O, H₂O₂, O₂O₃, NH₃, H₂S, H₂Se, PH₃, AsH₃, C₂H₄, and Si₂H₆.

The exposure temperature in the step (a) is not particularly limited, but is, for example, 100° C. or higher and 800° C. or lower, preferably 150° C. or higher and 650° C. or lower, more preferably 180° C. or higher and 500° C. or lower, and still more preferably 200° C. or higher and 375° C. or lower.

The exposure temperature in the step (b) is not particularly limited and may be a temperature substantially equal to or higher than the exposure temperature in the step (a). The film to be formed according to the ALD method is not particularly limited, and examples thereof include a film containing a pure element (such as Si, Cu, Ta, or W), a film containing an oxide (such as SiO₂, GeO₂, HfO₂, ZrO₂, Ta₂O₅, TiO₂, Al₂O₃, ZnO, SnO₂, Sb₂O₅, B₂O₃, In₂O₃, or WO₃), a film containing a nitride (such as Si₃N4, TiN, AlN, BN, GaN, or NbN), a film containing a carbide (such as SiC), a film containing a sulfide (such as CdS, ZnS, MnS, WS₂, or PbS), a film containing a selenide (such as CdSe or ZnSe), a film containing a phosphide (GaP or InP), a film containing an arsenide (such as GaAs or InAs), and a mixture thereof.

### EXAMPLES

Hereinafter, the present invention will be described in more detail with reference to examples, but the present invention is not limited to these examples.

### Synthesis Example 1: Synthesis of Octadecanohydroxamic Acid

Hydroxylamine hydrochloride (1.40 g, 20.1 mmol) and potassium carbonate (4.80 g, 34.7 mmol) were put into a 500 mL three-necked flask, and ethyl acetate (100.7 g) and water (66.0 g) were added thereto in an ice bath. After dissolution, a solution of stearoyl chloride (5.04 g, 16.6 mmol) in ethyl acetate (32.0 g) was added thereto using a dropping funnel and the mixture was allowed to react at room temperature for 18 hours. The organic layer was taken out, a 1 wt % HCl aqueous solution (108.1 g) was added thereto, and the mixture was stirred at room temperature for 25 minutes. The aqueous layer was extracted twice with ethyl acetate. The organic layers were combined and washed 4 times with 180 g of water. The organic layer was dried and solidified using a rotary evaporator, thereby obtaining a crude product (4.48 g). 2.48 g of the crude product was added to 247.76 g of methanol, dissolved therein under reflux, and filtered, and the filtrate was allowed to be naturally cooled at room temperature for 1 hour and filtered. The filter was recrystallized from methanol again, thereby obtaining white acicular crystals of octadecanohydroxamic acid (0.98 g).

The obtained compound was subjected to NMR measurement, and the structure was identified based on the following results.

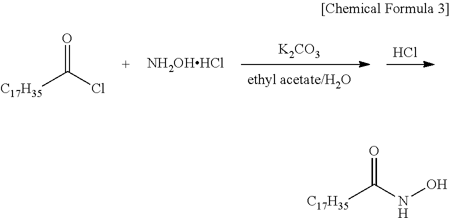

¹H-NMR (DMSO, 400 MHz): δ (ppm)=0.85 (t, CH₃, 3H), 1.10-1.35 (m, CH₂, 28H), 1.45 (t, CH₂, 2H), 1.92, 2.23 (t, CH₂, 2H), 8.65, 8.97 (s, NH, 1H), 9.72, 10.35 (s, OH, 1H)

### Preparation of Surface Treatment Agent

Each component listed in Table 1 was mixed to prepare a surface treatment agent of each example.

TABLE 1

| Surface treatment | | |
|---|---|---|
| agent | Compound | Solvent |
| A | Benzohydroxamic acid | PGME |
| | [0.5 mol/L] | |
| B | Octanohydroxamic acid | PGME |
| | [0.5 mol/L] | |
| C | Octadecanohydroxamic acid | PGME |
| | [0.75 mol/L] | |
| D | — | PGME |

As benzohydroxamic acid and octanohydroxamic acid, products manufactured by Tokyo Chemical Industry Co., Ltd. were used. Propylene glycol monomethyl ether (PGME) was used as the solvent.

### Examples 1 to 3 and Comparative Example 1

<Surface Treatment>

Using the surface treatment agents A to D prepared in the above-described manner, the surface treatment was performed on a W substrate, a Cu substrate, a Co substrate, an Al₂O₃ substrate, a SiO₂ substrate, a TiN substrate, and a Ru substrate according to the following method.

Specifically, the pretreatment was performed by immersing each substrate in a HF aqueous solution having a concentration of 0.5% by mass at 25° C. for 1 minute. After the pretreatment, the substrate was washed with ion exchange distilled water for 1 minute. The substrate after being washed with water was dried by nitrogen stream.

The surface treatment was performed on the substrate by immersing each of the dried substrates in the surface treatment agent of each example under the surface treatment conditions of 60° C. for 60 minutes. The surface-treated substrate was washed with isopropanol for 1 minute and further washed with ion exchange distilled water for 1 minute. The washed substrate was dried by nitrogen stream to obtain a surface-treated substrate.

### Measurement of Contact Angle of Water

The contact angle of water was measured for each substrate after the surface treatment described above.

The contact angle of water was measured by dropping pure water droplets (2.0 μL) on the surface of the surface-treated substrate using Dropmaster700 (manufactured by Kyowa Interface Science Co., Ltd.), and the contact angle obtained 2 seconds after the dropping of the droplets was used for the measurement. The results are listed in Table 2.

TABLE 2

| | Surface | | | | | | | |
|---|---|---|---|---|---|---|---|---|
| | treatment | Contact angle (°) | | | | | | |
| | agent | W | Cu | Co | Al₂O₃ | SiO₂ | TiN | Ru |
| Example 1 | A | 9 | 63 | 95 | 41 | 18 | 49 | 52 |
| Example 2 | B | 14 | 107 | 116 | 79 | 4 | 115 | 62 |
| Example 3 | C | 84 | 115 | 124 | 107 | 25 | 119 | 83 |
| Comparative | D | 4 | 24 | 21 | 15 | 24 | 5 | 35 |
| Example 1 | | | | | | | | |

Based on the results listed in Table 2, it was confirmed that in Examples 1 to 3 in which the surface treatment agents A to C were used, the contact angles of various substrates other than SiO₂ were improved as compared with Comparative Example 1 in which the surface treatment agent D was used.

### Examples 4 to 9

<Heat Treatment>

Each substrate which had been subjected to the surface treatment in the section of <surface treatment> described above was subjected to a heat treatment in a nitrogen atmosphere under the heating conditions listed in Table 3.

### Measurement of Contact Angle of Water

The contact angle of water was measured for each substrate after the heat treatment described above.

The contact angle of water was measured by dropping pure water droplets (2.0 μL) on the surface of the surface-treated substrate using Dropmaster700 (manufactured by Kyowa Interface Science Co., Ltd.), and the contact angle obtained 2 seconds after the dropping of the droplets was used for the measurement. The results are listed in Table 3.

TABLE 3

| | Surface | | | | | | | | |
|---|---|---|---|---|---|---|---|---|---|
| | treatment | Heating | Contact angle (°) | | | | | | |
| | agent | condition | W | Cu | Co | Al₂O₃ | SiO₂ | TiN | Ru |
| Example 4 | A | 100° C. for | 22 | 60 | 94 | 70 | 20 | 69 | 63 |
| | | 20 minutes | | | | | | | |
| Example 5 | A | 200° C. for | 36 | 55 | 28 | 61 | 21 | 32 | 55 |
| | | 20 minutes | | | | | | | |
| Example 6 | B | 100° C. for | 44 | 94 | 121 | 92 | 6 | 117 | 74 |
| | | 20 minutes | | | | | | | |
| Example 7 | B | 200° C. for | 30 | 64 | 33 | 79 | 7 | 54 | 41 |
| | | 20 minutes | | | | | | | |
| Example 8 | C | 100° C. for | 81 | 106 | 118 | 115 | 31 | 119 | 87 |
| | | 20 minutes | | | | | | | |
| Example 9 | C | 200° C. for | 59 | 77 | 100 | 108 | 30 | 57 | 56 |
| | | 20 minutes | | | | | | | |

Based on the results listed in Table 3, it was confirmed that in Examples 4 and 5 in which the surface treatment agent A was used, the contact angles with respect to the Cu substrate, the Co substrate, the TiN substrate, and the Ru substrate were decreased by the heat treatment carried out at 200° C. for 20 minutes, and thus the surface treatment agent was able to be desorbed from the substrate by a high temperature heat treatment.

It was confirmed that in Examples 6 and 7 in which the surface treatment agent B was used, the contact angles with respect to the Cu substrate, the Co substrate, the TiN substrate, and the Ru substrate were decreased by the heat treatment carried out at 200° C. for 20 minutes, and thus the surface treatment agent was able to be desorbed from the substrate by a high temperature heat treatment.

It was confirmed that in Examples 8 and 9 in which the surface treatment agent C was used, the contact angles with respect to the W substrate, the Cu substrate, the Co substrate, the TiN substrate, and the Ru substrate were decreased by the heat treatment carried out at 200° C. for 20 minutes, and thus the surface treatment agent was able to be desorbed from the substrate by a high temperature heat treatment.

While preferred embodiments of the invention have been described and illustrated above, it should be understood that these are exemplary of the invention and are not to be considered as limiting. Additions, omissions, substitutions, and other modifications can be made without departing from the spirit or scope of the invention. Accordingly, the invention is not to be considered as being limited by the foregoing description and is only limited by the scope of the appended claims.

What is claimed is:

1. A surface treatment agent used for treating a substrate which has a surface having two or more regions made of materials that are different from each other, the agent comprising:
a compound (H) represented by Formula (H-1), 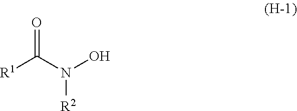
wherein R¹ represents a linear or branched alkyl group having 1 to 30 carbon atoms which may have a substituent, a linear or branched fluorinated alkyl group having 1 to 30 carbon atoms which may have a substituent, an aromatic hydrocarbon group which may have a substituent, or a cycloalkyl group having 3 to 12 carbon atoms which may have a substituent, and R² represents a hydrogen atom, a linear or branched alkyl group having 1 to 8 carbon atoms which may have a substituent, or a cycloalkyl group having 3 to 12 carbon atoms which may have a substituent.

2. The surface treatment agent according to claim 1, wherein at least one of the two or more regions has a metal surface.

3. A surface treatment method for a substrate which has a surface having two or more regions made of materials that are different from each other, the method comprising exposing the surface to the surface treatment agent according to claim 1.

4. The surface treatment method according to claim 3, wherein at least one of the two or more regions has a metal surface.

5. A region selective film formation method for a surface of a substrate, the method comprising:
treating the surface of the substrate using the surface treatment method according to claim 3; and
forming a film on the surface of the substrate, which has been subjected to the surface treatment, using an atomic layer deposition method,
wherein an amount of a material of the film to be deposited region-selectively varies.
